(19) United States
(12) Patent Application Publication
(10) Pub. No.: US 2004/0132104 A1
(43) Pub. Date: Jul. 8, 2004

(54) Vitamin D assay

(76) Inventors: James L. Sackrison, Minnetonka, MN (US); Andrew Miller, New Brighton, MN (US); John Kamerud, Carmel, IN (US); Diana L. Ersfeld, St. Paul, MN (US); Gregory T. Olson, Lakeland, MN (US); Gordon D. MacFarlane, Minneapolis, MN (US)

Correspondence Address: POPOVICH, WILES & O'CONNELL, PA, 650 THIRD AVENUE SOUTH, SUITE 600, MINNEAPOLIS, MN, 55402, US

(21) Appl. No.: 10/706,567

(22) Filed: Nov. 12, 2003

Related U.S. Application Data
(60) Provisional application No. 60/438,385, filed on Jan. 7, 2003.

(51) Int. Cl. G01N 33/53

(52) U.S. Cl. 435/7.1

(57) ABSTRACT

A method of assaying a sample of blood or blood components for the presence of 25-hydroxy-vitamin D comprising: (a) lowering the pH of the sample to 5.5 or less to dissociate the 25-hydroxy-vitamin D from vitamin D binding proteins; and (b) determining the concentration of 25-hydroxy-vitamin D in the sample. The vitamin D binding proteins are not removed from the sample.

This application claims the benefit of provisional application Serial No. 60/438,385, filed Jan. 7, 2003, the contents of which are hereby incorporated herein by reference.

### FIELD OF THE INVENTION

The present invention relates to a method of assaying a biological sample for the presence of Vitamin D or Vitamin D metabolites. In particular, the invention relates to a method for assaying blood or blood components for the presence of 25-hydroxy-vitamin D.

### BACKGROUND OF THE INVENTION

The existence of a Vitamin D endocrine system is generally accepted as being responsible for the conversion of Vitamin D (calciferol) into several active forms. The 25 hydroxylation of Vitamin D in the liver is the initial step in Vitamin D activation and produces the major circulating form of Vitamin D, 25-hydroxy-vitamin D (also 25 hydroxylcalciferol or 25-OH Vitamin D). It is now clear that the production of 25-OH Vitamin D in the liver is not significantly regulated. 25-OH Vitamin D is primarily dependent on substrate concentration. As the predominant circulating form of Vitamin D in the normal population, 25-OH Vitamin D measurement in the blood is an excellent index of Vitamin D status. The measurement of 25-OH Vitamin D is becoming increasingly important in the management of patients with various disorders of calcium metabolism associated with rickets, neonatal hypocalcemia, pregnancy, nutritional and renal osteodystrophy, hypoparathyroidism, and postmenopausal osteoporosis.

Two principal forms of 25-OH Vitamin D are cholcalciferol (25-OH Vitamin D₃) and ergocalciferol (25-OH Vitamin D₂). 25-OH Vitamin D₃ is derived mainly from the action of ultraviolet light on the skin. 25-OH Vitamin D₂ is derived mainly from dietary sources. Since these two compounds provide contributions to the overall Vitamin D status of the individual, it is important that both forms are measured equally. A great deal of research has provided information about circulating levels of 25-OH Vitamin D metabolites and their physiological significance.

Vitamin D and its various metabolites primarily are transported through the blood bound to Vitamin D binding protein. Although Vitamin D binding protein is the most important carrier for Vitamin D and its metabolites, certain other proteins also transport these chemicals and these proteins collectively are the vitamin D binding proteins. Vitamin D binding protein has an affinity for 25-OH Vitamin D of 5×10⁸ M⁻¹, and is present in plasma at concentrations of 4 to 8 μM. Due to the relatively high plasma concentration of the Vitamin D binding protein, which has an affinity similar to that of antibodies, the 25-OH Vitamin D must be dissociated from the binding protein to make it available for analysis in the sample. Historical methods for accomplishing this dissociation rely on denaturing the Vitamin D binding protein with organic solvents (sometimes preceded by the use of ammonium sulfate to precipitate the Vitamin D binding protein). The Vitamin D binding proteins are then removed from the assay to enable antibody binding to Vitamin D.

Though low pH dissociation of ligands from proteins is a known method of release, this method has not been successfully applied in an assay for 25-OH Vitamin D. The present invention provides a direct measurement of Vitamin D without the removal of Vitamin D binding proteins using low pH dissociation of Vitamin D from the Vitamin D binding proteins.

### SUMMARY OF THE INVENTION

The invention provides a method of assaying a sample of blood or blood components for the presence of 25-hydroxy-vitamin D comprising: (a) lowering the pH of the sample to 5.5 or less to dissociate the 25-hydroxy-vitamin D from vitamin D binding proteins; and (b) determining the concentration of 25-hydroxy-vitamin D in the sample, wherein the vitamin D binding proteins are not removed from the sample.

Additional features and advantages of the invention are set forth in the description which follows and in part will be apparent from the description. The objectives and other advantages of the invention will be realized and attained by the Vitamin D assay as particularly pointed out in the written description and claims.

It is to be understood that both the foregoing general description and the following detailed description are exemplary and explanatory and are intended to provide further explanation of the invention as claimed.

### DETAILED DESCRIPTION OF THE INVENTION.

The invention provides a method of assaying a sample of blood or blood components for the presence of 25-hydroxy-vitamin D comprising: (a) lowering the pH of the sample to 5.5 or less to dissociate the 25-hydroxy-vitamin D from vitamin D binding proteins; and (b) determining the concentration of 25-hydroxy-vitamin D in the sample, wherein the vitamin D binding proteins are not removed from the sample. In embodiments of the invention, the pH of the sample is lowered to 5 or less, 4.5 or less, 4 or less, or 3 or less. Preferably, the pH of the sample is lowered to be in the range of from 2 to 5.5, more preferably from 4.0 to 4.5.

In preferred embodiments, the pH of the sample is lowered to 5.5 or less by adding a buffer having a pH of less than 5.5; lowered to 5 or less by adding a buffer having a pH of less than 5; lowered to 4.5 or less by adding a buffer having a pH of less than 4.5; lowered to 4 or less by adding a buffer having a pH of 4 or less; or lowered to 3 or less by adding a buffer having a pH of less than 3. In a preferred embodiment, the pH of the sample is lowered to be in the range of 2 to 5.5 by adding a buffer. In another preferred embodiment, the buffer is a citrate, citrate phosphate, or acetate buffer. The pH of the buffer can be reduced to 5.5 or less by adding HCl to the buffer. In a preferred embodiment, the sample of blood or blood components is serum or plasma.

In a preferred embodiment, the concentration of 25-hydroxy-vitamin D is determined by immunoassay. In another preferred embodiment, no precipitate is formed. In still another preferred embodiment, no ammonium sulfate is used. In another preferred embodiment, no precipitate is formed, and no ammonium sulfate is used.

The concentration of 25-OH Vitamin D is determined by any one of a number of methods involving various formats and signal detection systems known to those of ordinary skill in the art. Such formats include, without limitation, competition assays, sandwich assays, displacement assays, etc., involving solid phases, antibody precipitation, etc. Such signal detection systems are illustrated by various immunoassays, such as radioimmunoassay, enzyme-linked immunoassay, fluorescence immunoassay, chemiluminescence immunoassay, high-sensitivity light scattering immunoassay, and fluorescence polarization immunoassay (see, eg., J. Clin. Immunoassay, 7(1): 64 et seq. (Spring 1984)). Labeled vitamin D analogues suitable for use as tracers in such methods are known. In a preferred embodiment, the detection method is a chemiluminescence immunoassay.

Chemiluminescent reagents are generally hydrophobic in character, as is 25-hydroxy-vitamin D. When conjugated together, the resulting tracer complex is also very hydrophobic, making its use in aqueous assay systems difficult. To overcome the combined hydrophobicity of the tracer complex, the linking chemistry includes polar functional groups to increase the solubility. In a preferred embodiment, the linker includes 2,2′-(Ethylenedioxy)diethylamine. In another preferred embodiment, the linker includes polyethylene glycol. In another preferred embodiment, the linker includes diamino cyclohexane. In another preferred embodiment, the linker includes dimethyl adipimidate. In yet another preferred embodiment, the linker includes a diamino C₃- to C₂-chain.

A preferred 25-OH Vitamin D assay (competitive principle) includes diluting the sample, standards and control samples, in a phosphate-citrate buffer pH 4.3. A volume of tracer and antibody coated magnetic particles are contacted with the diluted sample and incubated at 37° C. for 20 minutes. After incubation the particles are separated by a magnet and washed three times with wash buffer. After excess tracer is removed the starter reagents are added. The Vitamin D concentration in sample and calibrators is measured via the chemiluminescence reaction induced. The light measured in relative light units is inversely proportional to the concentration of 25-OH Vitamin D. The amount of 25-OH Vitamin D present in the original sample is calculated by comparing the relative light units in the sample with a standard curve generated by assaying calibrators of known amounts of 25-OH Vitamin D.

The following examples serve to illustrate the present invention and are not intended to limit its scope.

25-OH Vitamin D Immunoassay

Materials

1. Magnetic Particles

One mL of 1% 0.27 μm paramagnetic particles were coated overnight at 37° C. with polyclonal goat anti-vitamin D (obtained from Heartland Assay Inc., Ames, Iowa. Particles were placed on a magnet, separated, and after washing three times with 0.1% Gelatin in phosphate buffered saline (PBS), 10 mL of 0.1% Gelatin-PBS was added and blocking of nonspecific-protein binding sites was achieved by incubation overnight at 37° C. The particles were then washed four times with PBS at room temperature.

2. Assay Buffer

The assay buffer can be a phosphate—citrate buffer containing 10% organic solvent, surfactants, and preservatives at pH 4.3. In the preferred embodiment, the organic solvent is acetonitrile at a concentration of 10% by volume. The assay buffer used in the examples below was a phosphate-citrate buffer at pH 4.3 containing 30 mM sodium phosphate dibasic, 60 mM citric acid, 50 mM sodium hydroxide, and 150 mM sodium chloride, 10% by volume acetonitrile, 0.1% by weight casein, 0.1% by volume CELPURE® P65 (available from Advanced Minerals Corporation, Goleta, Calif.), and 0.1% by volume ProCling 300 (available from Sigma-Aldrich, Inc., St. Louis, Mo.).

3. Tracer Conjugate

This conjugate was prepared by cross-linking Vitamin D and amino-butyl-ethyl isoluminol (ABEI) with a 2,2′-(Ethylenedioxy)diethylamine linker. Specifically, a solution of Vitamin D-NHS ester (5 mg/mL in ethyl acetate) was added to a solution of 2,2′-(Ethylenedioxy)diethylamine at 20×molar excess and reacted at room temperature for 1 hour. The reactants were purified by C₁₈ reverse phase HPLC using a gradient with H₂O—0.1% Trifluoroacetic acid (TFA) and Acetonitrile (ACN)-0.1% TFA. The desired product (25 OH Vitamin D—2,2′-(Ethylenedioxy)diethylamine) was then lyophlized. The resulting solid was dissolved in 250 μL N,N-Dimethylformamide (DMF). ABEI-NHS (9 mg; 2×molar excess) in 250 μL DMF was added, followed by 25 μL triethanolamine (TEA) and allowed to stir for 18 to 24 hours at room temperature, protected from light. The desired product was then purified using the HPLC gradient described above.

4. Calibrators

25-Hydroxyvitamin D calibrators were prepared from processed equine serum (to remove endogenous 25-OH Vitamin D) at concentrations of 0, 5, 12, 20, 40, and 100 ng/mL 25-OH Vitamin D.

5. Starter Reagents

Starter reagents were obtained from Byk-Sangtec, Dietzenbach, Germany (Catalog # 9319102). These reagents consist of a catalyst and basic reagent to initiate the chemiluminescent reaction.

6. Wash Reagents

Wash reagents were obtained from Byk-Sangtec, Dietzenbach, Germany (Catalog #9319100). These reagents consist of a generic buffer with surfactants.

Protocol 1

The procedure of the assay was as follows. To the assay cuvette (approximately 400 μL) was added 25 μL of serum or plasma. Two hundred twenty μL of assay buffer, 20 μL of tracer conjugate (previously titered to targeted signal response) and 20 μL of magnetic particles at 0.25% solids were added to the assay cuvette. The assay cuvette was incubated for 30 minutes at 37° C. After incubation, the particles were separated by a magnet and washed three times with wash buffer. After excess tracer was removed, the starter reagents were added. The Vitamin D concentrations in the sample and calibrators were measured via the induced chemiluminescence reaction. The light measured in relative light units was inversely proportional to the concentration of 25-OH Vitamin D. The amount of 25-OH Vitamin D present in the original sample was calculated by comparing the relative light units in the sample with a standard curve generated by assaying calibrators of known amounts of 25-OH Vitamin D.

TABLE 1
Typical Results

| | % Bound/Bound at |
|---|---|
| ng/mL | Zero Concentration |
| 0 | 100 |
| 5 | 96 |
| 12 | 84 |
| 20 | 77 |
| 40 | 67 |
| 100 | 38 |

The average calculated analytical sensitivity was <2.0 ng/mL. Correlation of this method against standard radioimmunoassay (DiaSorin, Stillwater, Minn., Catalog #68100E) using 183 patient samples analyzed by linear regression resulted in a line with slope of 0.81, intercept of 2.6 ng/mL, and a correlation coefficient of 0.92. When analyzed by Student's t test, the resulting p value was 0.286, indicating no significant difference between the two methods.

Protocol 2

Alternatively, the protocol may be performed in a two step manner as described below. To the assay cuvette were added 25 μL of sample, 220 μL of assay buffer, and 20 μL of magnetic particles at 0.25% solids. This mixture was incubated for 20 minutes at 37° C., then washed three times. Subsequently, 20 μL of tracer conjugate and 220 μL of assay buffer were added for an additional 10 minute incubation at 37° C. The particles were then washed again, starter reagents added, and the resulting light emission recorded for 3 seconds. Typical results were similar to Protocol 1. The average calculated analytical sensitivity was <2.0 ng/mL. Correlation of this method against the standard radioimmunoassay described above using 73 patient samples analyzed by linear regression resulted in a line with slope of 1.13, intercept of −1.8 ng/mL, and a correlation coefficient of 0.94.

The above descriptions are provided for the purpose of describing embodiments of the invention and are not intended to limit the scope of the invention in any way. It will be apparent to those skilled in the art that various modifications and variations can be made in the vitamin D assay without departing from the spirit or scope of the invention. Thus, it is intended that the present invention cover the modifications and variations of this invention provided they come within the scope of the appended claims and their equivalents.

What is claimed is:

1. A method of assaying a sample of blood or blood components for the presence of 25-hydroxy-vitamin D comprising:
(a) lowering the pH of the sample to 5.5 or less to dissociate the 25-hydroxy-vitamin D from vitamin D binding proteins; and
(b) determining the concentration of 25-hydroxy-vitamin D in the sample,
wherein the vitamin D binding proteins are not removed from the sample.

2. The method of claim 1, wherein the pH of the sample is lowered to 5 or less.

3. The method of claim 1, wherein the pH of the sample is lowered to 4.5 or less.

4. The method of claim 1, wherein the pH of the sample is lowered to 4 or less.

5. The method of claim 1, wherein the pH of the sample is lowered to 3 or less.

6. The method of claim 1, wherein the pH of the sample is lowered to be in the range of from 2 to 5.5.

7. The method of claim 1, wherein the pH of the sample is lowered to be in the range of from 4.0 to 4.5.

8. The method of claim 1, wherein the pH of the sample is lowered to 5.5 or less by adding a buffer having a pH of less than 5.5.

9. The method of claim 8, wherein the buffer is a citrate, citrate phosphate, or acetate buffer.

10. The method of claim 1, wherein the concentration of 25-hydroxy-vitamin D is determined by immunoassay.

11. The method of claim 1, wherein the sample of blood or blood components is serum or plasma.

12. The method of claim 1, wherein no precipitate is formed.

13. A method of claim 10, wherein a vitamin D tracer is used in the immunoassay.

14. A method of claim 13, wherein the vitamin D tracer is ABEI conjugated to 25-hydroxy-vitamin D.

15. A method of claim 13, wherein the vitamin D tracer is ABEI conjugated to a 22-carboxylic acid derivative of 25-hydroxy-vitamin D by a 2,2′-(Ethylenedioxy)diethylamine linker.

16. A method of claim 13, wherein the vitamin D tracer is ABEI conjugated to a 22-carboxylic acid derivative of 25-hydroxy-vitamin D by a polyethylene glycol linker.

17. A method of claim 13, wherein the vitamin D tracer is ABEI conjugated to a 22-carboxylic acid derivative of 25-hydroxy-vitamin D by a dimethyl adipimidate linker.

18. A method of claim 13, wherein the vitamin D tracer is ABEI conjugated to a 22-carboxylic acid derivative of 25-hydroxy-vitamin D by a diamino cyclohexane linker.

19. A method of claim 13, wherein the vitamin D tracer is ABEI conjugated to a 22-carboxylic acid derivative of 25-hydroxy-vitamin D by a diamino C₃- to C₁₂-chain linker.
